(19) United States
(12) Patent Application Publication
(10) Pub. No.: US 2014/0213964 A1
(43) Pub. Date: Jul. 31, 2014

(54) Breast Pump Adapter For Bag

(71) Applicant: THUNDERBIRD GLOBAL ENTERPRISES, LLC, Atlanta, GA (US)

(72) Inventor: Nossi Taheri, Atlanta, GA (US)

(73) Assignee: Thunderbird Global Enterprises, LLC, Atlanta, GA (US)

(21) Appl. No.: 13/755,734

(22) Filed: Jan. 31, 2013

(51) Int. Cl. A61M 1/06

(52) U.S. Cl. CPC A61M 1/06
USPC 604/74

(57) ABSTRACT

A breast pump adapter including a body, hooks, and wings. The body includes a top end that is configured to attach to a nozzle of a breast pump. The hooks extend outwardly from the body. Each hook is configured to be received in an aperture of a bag. The wings extend outwardly from the body. Each wing tapers from the body to a respective distal end.

### TECHNICAL FIELD

The technical field relates generally to adapters that configure a breast pump for use with a bag.

### BACKGROUND

Adapters are used to configure a breast pump for use with a bag (e.g., as opposed to a bottle). One problem with some current adapters is that the bag falls off the adapter during the process of filling the bag. Another problem with current adapters is that, during the process of filling the bag, the adapter sprays milk into the zipper of the bag or milk splashes into the zipper.

If milk gets in the zipper, the zipper does not completely close when the bag is placed in the freezer for storage. When the bag is subsequently thawed, the zipper leaks. A need exists in the industry to address the aforementioned deficiencies and inadequacies.

### SUMMARY

The various embodiments of the present disclosure overcome the shortcomings of the prior art by providing a breast pump adapter including a body, hooks, and wings. The body includes a top end that is configured to attach to a nozzle of a breast pump. The hooks extend outwardly from the body. Each hook is configured to be received in an aperture of a bag. The wings extend outwardly from the body. Each wing tapers from the body to a respective distal end.

In some embodiments, the top end includes threads.

In some embodiments, the body includes a bottom end and a channel. The channel includes a top opening at the top end and a bottom opening at the bottom end. In certain of these embodiments, the channel tapers from the top opening to the bottom opening, the bottom opening has an oval shape, and the top opening is circular.

In certain embodiments, a distance between one of the first and the second hook and one of the first wing and the second wing is greater than a distance between an aperture and a closer band of a bag.

In certain embodiments, the body, the first wing, and the second wing, together, have a shape that is substantially that of an opening of a milk bag.

In certain embodiments, the body, the first wing, and the second wing, together, have a shape that is substantially a pointed oval.

In certain embodiments, the position of the first wing and the second wing is further from the top end than the position of the first hook and the second hook.

In certain embodiments, the first wing and the second wing include a support.

In certain embodiments, the first wing and the second wing are on opposed sides of the body.

In certain embodiments, the first wing and the second wing are positioned to be substantially perpendicular to the first hook and the second hook.

In certain embodiments, the first wing and the second wing extend substantially horizontally. In other embodiments, the first wing and the second wing extend at an upward angle or a downward angle.

In certain embodiments, the first hook and the second hook include a supporting portion and the retaining portion. The supporting portion is configured to support an edge of an aperture of a bag and the retaining portion is configured to retain a bag on the respective supporting portion.

In certain embodiments, a supporting portion includes a curved upper surface.

In certain embodiments, a retaining portion includes a curved upper edge.

In certain embodiments, a retaining portion includes at least one protrusion extending outwardly from the edge of the retaining portion.

The foregoing has broadly outlined some of the aspects and features of the various embodiments, which should be construed to be merely illustrative of various potential applications of the disclosure. Other beneficial results can be obtained by applying the disclosed information in a different manner or by combining various aspects of the disclosed embodiments. Accordingly, other aspects and a more comprehensive understanding may be obtained by referring to the detailed description of the exemplary embodiments taken in conjunction with the accompanying drawings, in addition to the scope defined by the claims.

### DESCRIPTION OF THE DRAWINGS

FIG. 1 is a side view of an exemplary breast pump.

FIG. 2 is a front view of the breast pump of FIG. 1.

FIG. 3 is a perspective view of an adapter of the breast pump of FIG. 1.

FIG. 4 is another perspective view of the adapter of FIG. 3.

FIG. 5 is a side elevational view of the adapter of FIG. 3.

FIG. 6 is another side elevational view of the adapter of FIG. 3.

FIG. 7 is a top view of the adapter of FIG. 3.

### DETAILED DESCRIPTION

As required, detailed embodiments are disclosed herein. It must be understood that the disclosed embodiments are merely exemplary of various and alternative forms. As used herein, the word “exemplary” is used expansively to refer to embodiments that serve as illustrations, specimens, models, or patterns. The figures are not necessarily to scale and some features may be exaggerated or minimized to show details of particular components. In other instances, well-known components, systems, materials, or methods that are know to those having ordinary skill in the art have not been described in detail in order to avoid obscuring the present disclosure. Therefore, specific structural and functional details disclosed herein are not to be interpreted as limiting, but merely as a basis for the claims and as a representative basis for teaching one skilled in the art.

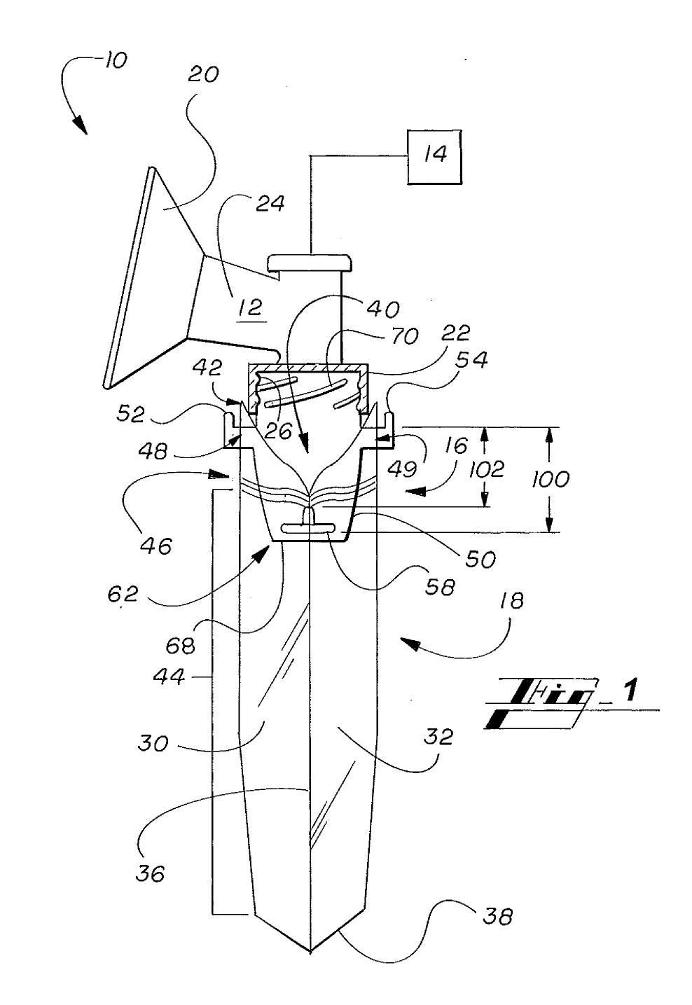

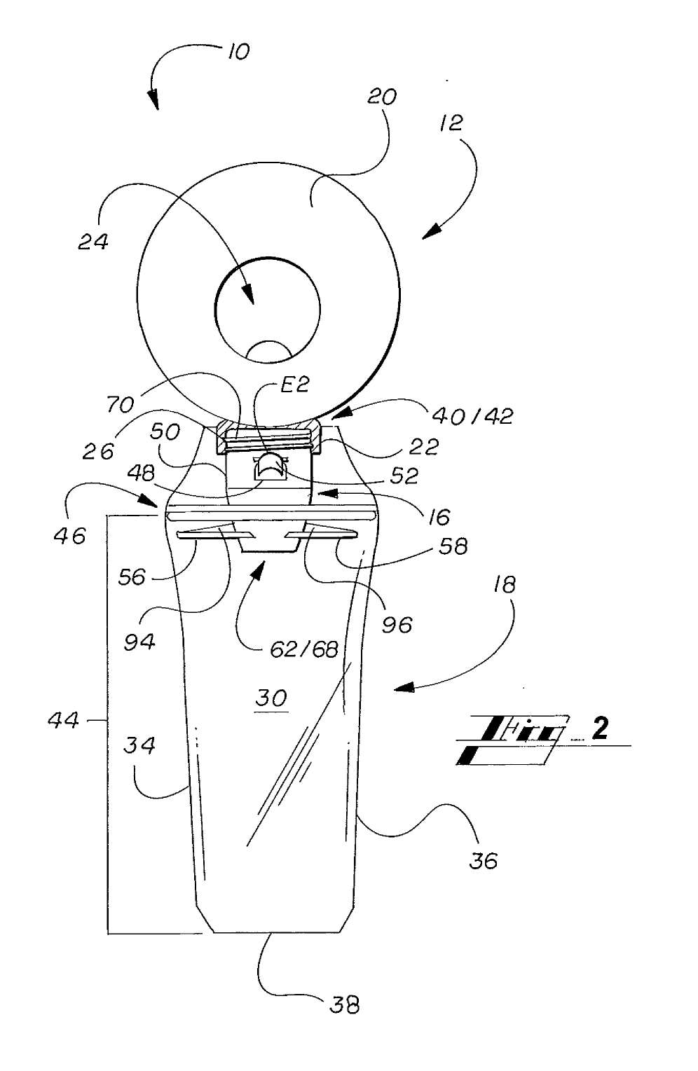

Referring to FIGS. 1 and 2, an exemplary breast pump 10 includes a breast-hood assembly 12, a pump 14, an adapter 16, and a milk-receiving bag 18. The breast-hood assembly 12, the pump 14, and the bag 18 are now briefly described. The adapter 16 is described in further detail below.

The breast-hood assembly 12 includes a cup 20 that is configured to interface with a breast and a nozzle 22 (shown in a cross-sectional view) that is configured to attach to the adapter 16. A channel 24 leads from the cup 20 to the nozzle 22. The inside surface of the nozzle 22 includes threads 26. Generally, the threads 26 in the nozzle 22 are universal for major brands of breast pumps.

Referring to FIG. 1, the pump 14 is connected to the channel 24 and is configured to control or apply pressure in the channel 24 of the breast-hood assembly 12 (e.g., to provide suction at the cup 20). The pump 14 can be a manual pump or a machine pump (e.g., an electrical pump). For purposes of teaching, the pump 14 is schematically illustrated.

Referring to FIGS. 1 and 2, the bag 18 includes a front wall 30 and a rear wall 32 that are connected to one another along side edges 34, 36. The bottom edges of the front and rear walls 30, 32 at least partially define a bottom end 38. The top edges of the front and rear walls 30, 32 are separable from one another to provide an opening 40 at a top end 42 through which milk or other pourable substances can enter and exit a containment region 44.

As will be used herein, the term longitudinal relates to the length of the bag 18, which is measured between the top end 42 and the bottom end 38. Likewise, the term transverse relates to the width of the bag 18, which is measured between the side edges 34, 36. These terms may be used to provide descriptions that pertain to alignment or direction of movement of elements with respect to the bag 18.

The bag 18 further includes a closure band 46. The closure band 46 includes one or more extruded zipper-type closures having elements that are integral to the walls of the bag 10. For example, each zipper-type closure includes a male profile that is integral to the front wall 30 and a female profile that is integral to the rear wall 32. The male profile and the corresponding female profile are capable of releasably engaging one another to close the opening 40 of the bag 18 so as to seal a pourable substance in the containment region 44.

The portions of the walls 30, 32 above the closure band 46 are referred to herein as lips. The lips include apertures 48, 49 that are configured to hang the bag 18 on the adapter 16 as described in further detail below. The exemplary apertures 48, 49 have a semi-circular shaped upper edge E1 (see FIG. 6).

Exemplary bags include a plastic bag as disclosed in U.S. Pat. No. 8,303,182, which is herein incorporated by reference. The adapter 16 is configured to be used with such bags.

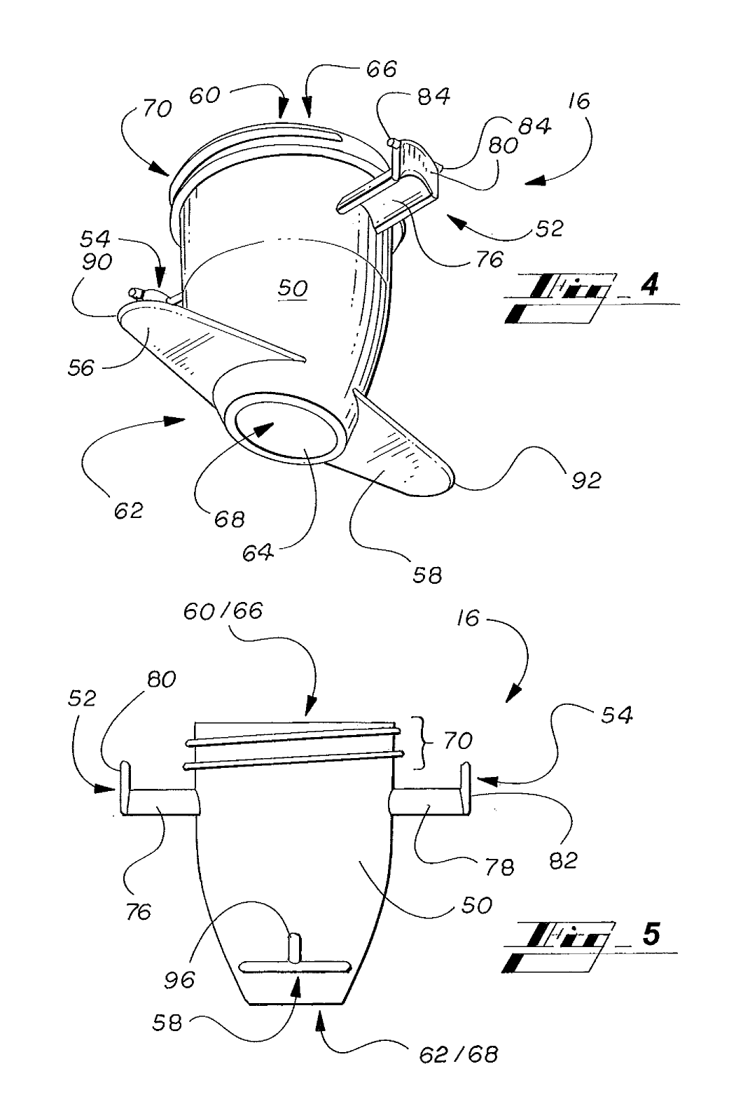

Referring to FIGS. 1-7, the adapter 16 is described in further detail. The adapter 16 includes a body 50, bag hooks 52, 54 and splash-resisting wings 56, 58.

The body 50 includes a top end 60, a bottom end 62, and a channel 64 leading through the body 50 from the top end 60 to the bottom end 62. The channel 64 includes a top opening 66 at the top end 60 and a bottom opening 68 at the bottom end 62. The channel 64 is tapered or narrows moving from the top end 60 to the bottom end 62.

The top end 60 of the adapter 16 is configured to attach to the nozzle 22 of the breast-hood assembly 12. Specifically, the top end 60 (and the top opening 66) has a circular shape so as to fit snugly in the nozzle 22. Threads 70 are positioned on the outside of the body 50 near the top end 60. The threads 70 are configured to interface with the threads 26 that are positioned on the inside of the nozzle 22.

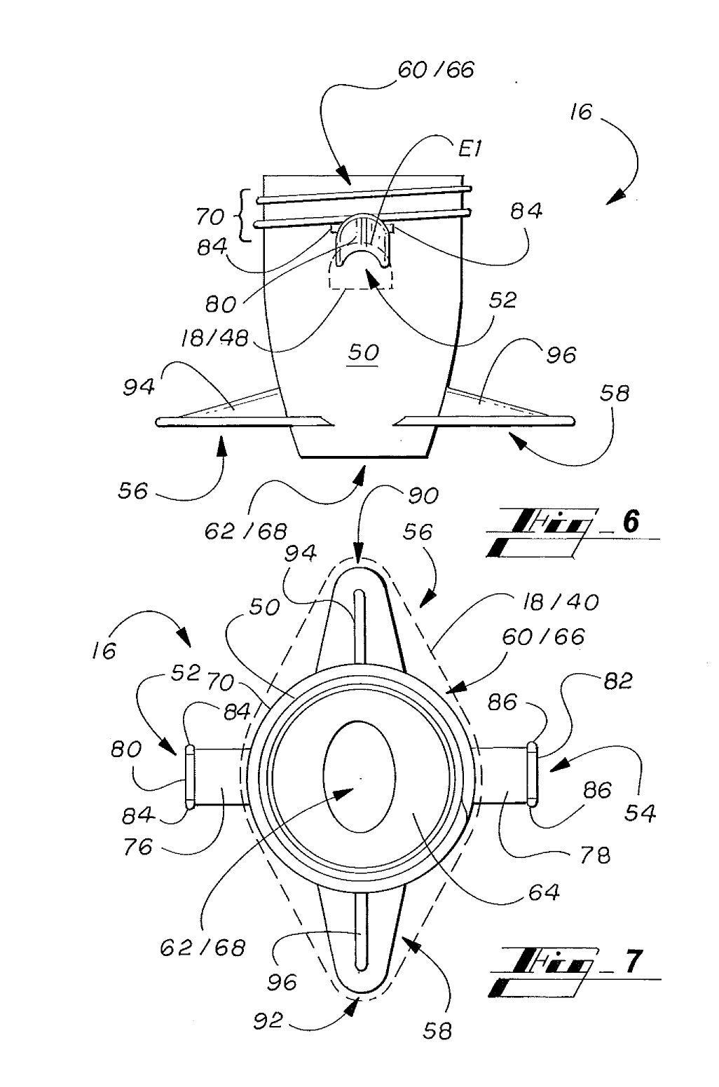

Referring to FIGS. 4 and 7, the bottom opening 68 at the bottom end 62 has an oval shape. The dimensions of the oval shape are determined so that when breast milk flows through the channel 64 of the adapter 16 and exits through the bottom opening 68, the oval-shaped bottom opening 68 directs the breast milk toward the walls of the milk bag 18. By directing the breast milk toward the walls of the milk bag 18, the oval-shaped bottom opening 68 reduces the amount of splash in the milk bag 18. For example, a circular-shaped bottom opening directs breast milk straight down to the bottom of the bag 18, which causes a splash. Splashing can cause milk to undesirably splash into the closure band 46, which can create leaking problems as described in further detail below.

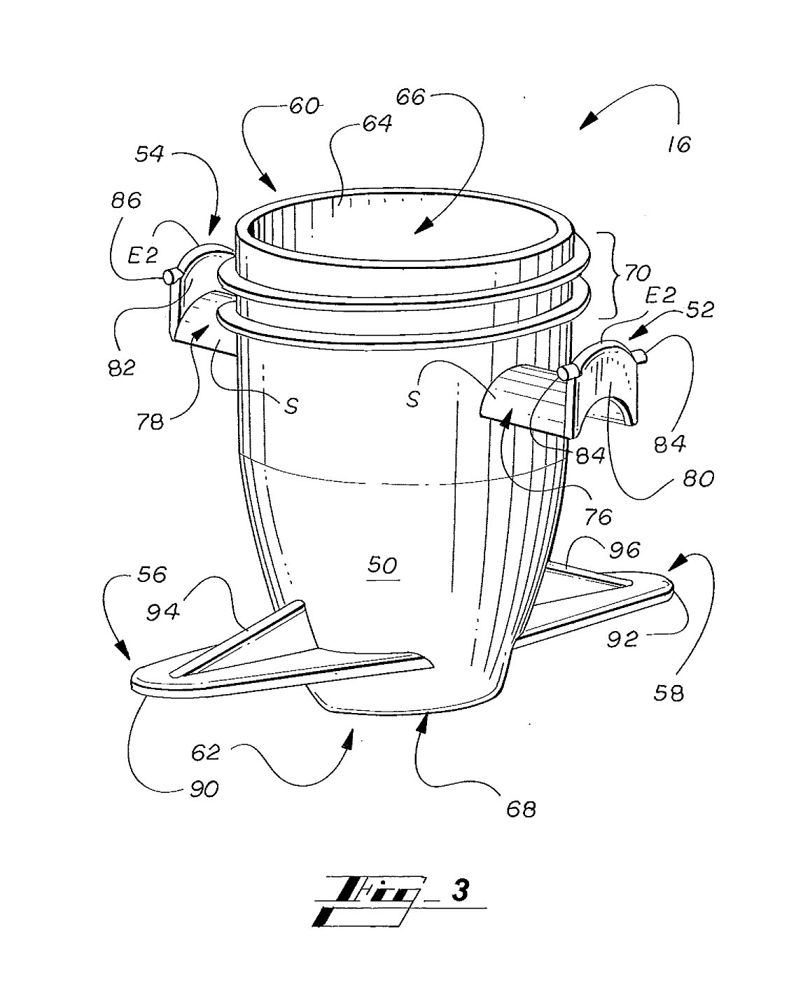

Referring to FIG. 3, the hooks 52, 54 are configured such that the bag 18 can be attached to the adapter 16 by the apertures 48, 49. The hooks 52, 54 include a supporting portion 76, 78, a retaining portion 80, 82, and protrusions 84, 86, respectively. The supporting portions 76, 78 are configured to provide a structure on which the bag 18 can hang and the retaining portions 80, 82 and protrusions 84, 86 are configured to retain the bag 18 on the supporting portions 76, 78.

In the illustrated embodiment, the supporting portions 76, 78 extend outwardly (e.g., substantially horizontally) from the body 50 and the retaining portions 80, 82 extend upwardly (e.g., substantially vertically) from the distal ends of the supporting portions 76, 78. The protrusions 84, 86 extend outwardly from an edge E2 of the retaining portions.

In addition, the upper facing surfaces S of the supporting portions 76, 78 and the upper edges of the retaining portions 80, 82 are shaped to work with the apertures 48, 49 in the bag 18. The upper facing surfaces S of the supporting portions 76, 78 have a semi-circular shape that matches the shape of the upper edge E1 of the apertures 48, 49. As such, the hanging force of the bag 18 is distributed over the upper edge E1 of the apertures 48, 49 to reduce the chance of tearing. In alternative embodiments, the upper facing surface S is substantially flat.

The retaining portions 80, 82 are shaped to match the shape of the apertures 48, 49 such that the retaining portions 80, 82 can move through the apertures 48, 49 without tearing the bag 18 (e.g., see FIG. 6 where the dashed line represents the aperture 48). The protrusions 84, 86 are dimensioned such that the apertures 48, 49 tightly fit over the retaining portions 80, 82. As such, the bag 18 does not unintentionally fall off the adapter 16.

The wings 56, 58 are positioned on the body 50 between the hooks 52, 54 and the bottom end 62. Alternatively described, the position of the wings 56, 58 on the body 50 is further from the top end 60 than the position of the hooks, 52, 54 on the body 50.

The wings 56, 58 extend outwardly (e.g., substantially horizontally as shown in FIG. 6). In alternative embodiments, the wings 56, 58 extend downward or upward at various angles.

Referring to FIG. 7, the direction of extension of the wings 56, 58 from the body 50 is substantially perpendicular to the direction of extension of the hooks 52, 54 from the body. The threads 70 are configured such that, when the adapter 16 is tightened in the nozzle 22, the hooks 52, 54 are aligned with the direction of the channel 24 through the cup 20 and the wings 56, 58 are substantially perpendicular to the direction of the channel 24 through the cup 20. As such, referring to FIG. 1, the front wall 30 of the bag 18 lays flat against the user during use.

Referring to FIG. 3, the wings 56, 58 include supports 94, 96 that connect the wings 56, 58 to the body 50. The supports 94, 96 reinforce the connection between the wings 56, 58 and the body 50 to prevent a break, for example, were the wings 56, 58 to catch on the bag 18 as the adapter 16 is inserted into or removed from a bag 18.

The wings 56, 58 are splash guards. When the bag 18 is attached to the adapter 16, the adapter 16 is positioned in the bag 18 such that the wings 56, 58 substantially close the opening 40 of the bag 18 to keep milk from splashing upwardly to the closure band 46 or out of the bag 18. Particularly, referring to FIG. 7, the wings 56, 58 have substantially the same shape as the shape of the opening 40 of the bag 18 when the bag 18 is opened to receive the adapter 16. The opening 40 of the bag 18 has a pointed oval shape and the wings 56, 58 taper to a point (e.g., taper from the connection to the body 50 toward a distal end 90, 92) to match the shape of the opening 40 of the bag 18.

Referring to FIGS. 1 and 2, the adapter 16 has a length and the wings 56, 58 have a position relative to the hooks 52, 54 such that milk that enters the bag 18 through the adapter 16 is prevented from splashing or flowing to the closure band 46. If milk gets into the closure band 46, when the milk is frozen in the bag 18 for storage, the frozen milk in the closure band 46 can prevent the bag 18 from fully closing and cause leakage upon thawing the bag 18.

A distance 100 between the hooks 52, 54 and the wings 56, 58 is greater than a distance 102 between the apertures 48, 49 and the closure band 46 such that the wings 56, 58 are positioned below the closure band 46 when the bag 18 is attached to the adapter 16. As such, the wings 56, 58 prevent milk from splashing up to the closure band 46. Further, the bottom opening 68 is positioned below the closure band 46.

According to an exemplary method of assembling the breast pump 10, the adapter 16 is attached to the breast-hood assembly 12 by threading the adapter 16 into the nozzle 22. The bag 18 positioned such that the adapter 16 is received in the opening 40 and the apertures 48, 49 are pulled over the retaining portions 80, 82 and rest on the supporting portions 76, 78 such that the bag 18 hangs on the hooks 52, 54. Thereby, the wings 56, 58 and the bottom opening 68 are positioned below the closure band 46. During operation of the breast pump 10, the pump 14 pulls milk into the containment region 44 of the bag 18 through the adapter 16 and the wings 56, 58 and the bottom opening 68 prevent splashing of the milk up to the closure band 46.

The above-described embodiments are merely exemplary illustrations of implementations that are set forth for a clear understanding of principles. Variations, modifications, and combinations may be made to the above-described embodiments may be made without departing from the scope of the claims. All such variations, modifications, and combinations are included herein by the scope of this disclosure and the following claims.

What is claimed is:

1. A breast pump adapter, comprising:
a body comprising a top end that is configured to attach to a nozzle of a breast pump;
a first hook and a second hook extending outwardly from the body, each of the first hook and the second hook being configured to be received in an aperture of a bag; and
a first wing and a second wing extending outwardly from the body, each of the first wing and the second wing tapering from the body to a respective distal end.

2. The breast pump adapter of claim 1, the top end comprising threads.

3. The breast pump adapter of claim 1, the body comprising a bottom end and a channel, the channel comprising a top opening at the top end and a bottom opening at the bottom end.

4. The breast pump adapter of claim 3, wherein the channel tapers from the top opening to the bottom opening.

5. The breast pump adapter of claim 3, wherein the bottom opening has an oval shape.

6. The breast pump adapter of claim 5, wherein the top opening is circular.

7. The breast pump adapter of claim 1, wherein a distance between one of the first and the second hook and one of the first wing and the second wing is greater than a distance between an aperture and a closer band of a bag.

8. The breast pump adapter of claim 1, the body, the first wing, and the second wing, together, having a shape that is substantially that of an opening of a milk bag.

9. The breast pump adapter of claim 1, the body, the first wing, and the second wing, together, having a shape that is substantially a pointed oval.

10. The breast pump adapter of claim 1, the position of the first wing and the second wing being further from the top end than the position of the first hook and the second hook.

11. The breast pump adapter of claim 1, each of the first wing and the second wing including a support.

12. The breast pump adapter of claim 1, the first wing and the second wing are on opposed sides of the body.

13. The breast pump adapter of claim 1, wherein the first wing and the second wing are positioned to be substantially perpendicular to the first hook and the second hook.

14. The breast pump adapter of claim 1, wherein the first wing and the second wing extend substantially horizontally.

15. The breast pump adapter of claim 1, wherein the first wing and the second wing extend at an upward angle.

16. The breast pump adapter of claim 1, wherein the first wing and the second wing extend at a downward angle.

17. The breast pump adapter of claim 1, each of the first hook and the second hook comprising a supporting portion and the retaining portion, each supporting portion being configured to support an edge of an aperture of a bag, each retaining portion being configured to retain a bag on the respective supporting portion.

18. The breast pump adapter of claim 17, each supporting portion comprising a curved upper surface.

19. The breast pump adapter of claim 17, each retaining portion comprising a curved upper edge.

20. The breast pump adapter of claim 17, each retaining portion comprising at least one protrusion extending outwardly from the edge of the retaining portion.
